(19) United States
(12) Patent Application Publication
(10) Pub. No.: US 2026/0224481 A1
(43) Pub. Date: Aug. 6, 2026

(54) METHODS AND MATERIALS FOR TREATING HYPOCAPNIA

(71) Applicant: Mayo Foundation for Medical Education and Research, Rochester, MN (US)

(72) Inventors: Jan Stepanek, Scottsdale, AZ (US); Michael J. Cevette, Elgin, AZ (US); Jonathan R. Tomshine, Rochester, MN (US); Caroline M. Murray, Saint Paul, MN (US); Joshua K. Strakos, Burnsville, MN (US)

(21) Appl. No.: 19/132,491

(22) PCT Filed: Dec. 13, 2023
(86) PCT No.: PCT/US2023/083896

§ 371 Date: May 23, 2025

Related U.S. Application Data
(60) Provisional application No. 63/432,202, filed on Dec. 13, 2022.

(51) Int. Cl. A61K 9/00; A61K 33/00

(52) U.S. Cl. CPC A61K 9/007; A61K 33/00

(57) ABSTRACT

This document provides systems, devices, and methods for treating hypocapnia. For example, systems, devices, and methods for delivering carbon dioxide (CO₂) to a user are provided.

### CROSS-REFERENCE TO RELATED APPLICATIONS

This application claims the benefit of U.S. Provisional Application Ser. No. 63/432,202, filed Dec. 13, 2022. The disclosure of the prior application is considered part of the disclosure of this application, and is incorporated in its entirety into this application.

### BACKGROUND

### 1. Technical Field

This document relates to methods and materials involved in treating hypocapnia, a state of decreased carbon dioxide within the body. For example, this document provides methods and materials for delivering carbon dioxide (CO₂) to a human to treat hypocapnia or compensate for a reduced level of CO₂.

### 2. Background Information

Hypocapnia is a condition where a mammal has a reduced level of carbon dioxide in the blood. It usually occurs as a result of excessive ventilation (e.g., increased depth of breathing and/or increased rate of breathing) that exceeds metabolic carbon dioxide production in the body.

### SUMMARY

This document provides methods and materials for treating hypocapnia. For example, this document provides methods and materials for delivering CO₂ to a human to treat hypocapnia or compensate for a reduced level of CO₂. As described herein, delivering CO₂ to a person suffering from hypocapnia can treat the hypocapnia or compensate for a reduced level of CO₂ and rapidly resolve the symptoms of hypocapnia. In some cases, having the ability to reduce hypocapnia and the symptoms of hypocapnia rapidly as described herein can allow a person to resume normal activities without needing additional medical care.

In general, one aspect of this document features a system for delivering carbon dioxide (CO₂) to a user, the system including a fluid receptacle defining an interior space to contain a fluid and a cap device configured to be coupled to the fluid receptacle. The cap device includes an inner shell, an outer shell that is rotatably coupled about the inner shell, a mouthpiece positioned on the outer shell, a vent extending through the outer shell, a first opening extending through the inner shell, and a second opening extending through the inner shell. The cap device can be configured to be positioned in an open position by rotating the outer shell relative to the inner shell to align the vent with the first opening and to align the mouthpiece with the second opening, wherein the mouthpiece is in fluid communication with the interior space of the fluid receptacle when the cap device is coupled to the fluid receptacle and in the open position. The cap device can be configured to be positioned in a closed position by rotating the outer shell relative to the inner shell to offset the vent and the mouthpiece from the first opening and the second opening, wherein the mouthpiece is not in fluid communication with the interior space of the fluid receptacle when the cap device is coupled to the fluid receptacle and in the closed position. The mouthpiece can be positioned on a sidewall of the outer shell and at least one of the first opening and the second opening can extend through a sidewall of the inner shell. The vent can be positioned on a top surface of the outer shell and at least one of the first opening and the second opening can extend through a top surface of the inner shell. The fluid receptacle can be at least partially filled with a liquid and the system can further include a composition that releases CO₂ for the user to inhale once the composition is placed within the liquid in the fluid receptacle. The composition can be a solid composition. The cap device can further include a sealing element configured to seal the cap device against the fluid receptacle. The cap device can further include a mesh positioned within and coupled to the inner shell. The fluid receptacle can be configured to be directly coupled to the cap device. The fluid receptacle can include a first set of threads and the cap device can include a second set of threads, wherein the second set of threads are configured to releasably engage the first set of threads. An outer diameter of a portion of the fluid receptacle containing the first set of threads can correspond to an inner diameter of a portion of the cap device that contains the second set of threads. The system can further include an adaptor configured to couple the fluid receptacle to the cap device. The fluid receptacle can include a first set of threads, the cap device can include a second set of threads, and the adaptor can include a third set of threads configured to releasably engage the first set of threads of the fluid receptacle and a fourth set of threads configured to releasably engage the second set of threads of the cap device. The third set of threads can be positioned on an inner surface of the adaptor and the fourth set of threads can be positioned on an outer surface of the adaptor. An outer diameter of a portion of the fluid receptacle containing the first set of threads can be smaller than an inner diameter of a portion of the cap device that contains the second set of threads.

An outer diameter of a portion of the fluid receptacle containing the first set of threads can be larger than an inner diameter of a portion of the cap device that contains the second set of threads. The adaptor can include a sealing element configured to seal the adaptor against the fluid receptacle. The system can further include a drinking straw extending through the cap device into the fluid receptacle, the drinking straw being configured to enable a user to drink the fluid inside the fluid receptacle. At least a portion of the inner shell of the cap device and at least a portion of the outer shell of the cap device can include a collapsible silicone material, and the cap device can be configured to deform into a collapsed state. The system can further include a tablet loader coupled to the fluid receptacle, the tablet loader being configured to house a one or more tablets that include a composition that releases CO₂ for the user to inhale once the composition is placed within the liquid in the fluid receptacle. The tablet loader can include a tablet housing, a spring configured to apply upwards force on the one or more tablets, a button operable to move one of the one or more tablets into the fluid receptacle, and a pair of elastomeric seals configured to fluidly seal the tablet housing from the fluid receptacle.

In another aspect, this document features a cap device configured to be coupled to a fluid receptacle for delivering carbon dioxide (CO₂) to a user, the cap device including an inner shell, an outer shell that is rotatably coupled about the inner shell, a mouthpiece positioned on the outer shell, a vent extending through the outer shell, a first opening extending through the inner shell, and a second opening extending through the inner shell. The cap device can be configured to be positioned in an open position by rotating the outer shell relative to the inner shell to align the vent with the first opening and to align the mouthpiece with the second opening. The cap device can be configured to be positioned in a closed position by rotating the outer shell relative to the inner shell to offset the vent and the mouthpiece from the first opening and the second opening. The mouthpiece can be positioned on a sidewall of the outer shell and at least one of the first opening and the second opening can extend through a sidewall of the inner shell. The vent can be positioned on a sidewall of the outer shell and at least one of the first opening and the second opening can extend through a sidewall of the inner shell. The vent can be positioned on a top surface of the outer shell and at least one of the first opening and the second opening can extend through a top surface of the inner shell. The cap device can further include a sealing element configured to seal the cap device against the fluid receptacle. The cap device can further include a mesh positioned within and coupled to the inner shell. The cap device can be configured to be directly coupled to the fluid receptacle. The cap device can further include a first set of threads configured to releasably engage a second set of threads on the fluid receptacle. An inner diameter of a portion of the cap device can correspond to an outer diameter of a portion of the fluid receptacle.

In another aspect, a method of delivering carbon dioxide (CO₂) to a user includes attaching a cap device to a fluid receptacle defining an interior space to contain a fluid, the cap device including a mouthpiece; placing the cap device in an open position, wherein the mouthpiece is in fluid communication with the interior space of the fluid receptacle when the cap device is coupled to the fluid receptacle and in the open position; and breathing into and out of the fluid receptacle through the mouthpiece of the cap device. The cap device can include an inner shell and an outer shell rotatably coupled about the inner shell, and placing the cap device in the open position can include rotating the outer shell relative to the inner shell. The cap device can include an opening extending through the inner shell; and placing the cap device in the open position can include rotating the outer shell relative to the inner shell to align the mouthpiece with the opening. The cap device can include a vent extending through the outer shell and a second opening extending through the inner shell; and placing the cap device in the open position can include rotating the outer shell relative to the inner shell to align the vent with the second opening. The fluid receptacle can include a first set of threads, the cap device can include a second set of threads, and attaching the cap device to the fluid receptacle can include releasably engaging the first set of threads of the fluid receptacle with the second set of threads of the cap device. Attaching the cap device to the fluid receptacle can include attaching the cap device and the fluid receptacle to an adaptor. The fluid receptacle can be at least partially filled with a liquid; and the method can further include, prior to attaching the cap device to the fluid receptacle, placing a composition that releases CO₂ when wetted into the liquid within the fluid receptacle. The method can further include, prior to attaching the cap device to the fluid receptacle, placing the cap device in a closed position, wherein the mouthpiece is not in fluid communication with the interior space of the fluid receptacle when the cap device is coupled to the fluid receptacle and in the closed position. The cap device can include an inner shell and an outer shell rotatably coupled about the inner shell; and placing the cap device in the closed position can include rotating the outer shell relative to the inner shell. The cap device can include an opening extending through the inner shell; and placing the cap device in the closed position can include rotating the outer shell relative to the inner shell to align the mouthpiece with the opening. The cap device can include a vent extending through the outer shell and a second opening extending through the inner shell; and placing the cap device in the close position can include rotating the outer shell relative to the inner shell to align the vent with the second opening.

In another aspect, a method of delivering carbon dioxide (CO₂) to a user includes placing a dry carbon dioxide saturated composition that releases CO₂ when heated within a receptacle defining an interior space; heating the dry carbon dioxide saturated composition inside the receptacle using a heating system at least partially disposed within the interior space to a threshold temperature to generate CO₂; and breathing into and out of the receptacle through a mouthpiece fluidly coupled to the interior space. The method can further include attaching a cap device that includes the mouthpiece to the receptacle. The method can further include mixing the generated CO₂ with ambient air inside the receptacle to produce a diluted CO₂ gas for inhalation. The dry carbon dioxide saturated composition can include one or more types of carbon dioxide saturated zeolites.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a cross sectional view of an example CO₂ delivery system.

FIG. 2 is a side view of the CO₂ delivery system of FIG. 1.

FIG. 3A is a perspective view of the CO₂ delivery system of FIG. 1 in an open position.

FIG. 3B is a perspective view of the CO₂ delivery system of FIG. 1 in a closed position.

FIG. 4A is a cross sectional view of the cap device of the CO₂ delivery system of FIG. 1 in an open position.

FIG. 4B is a cross sectional view of the cap device of the CO₂ delivery system of FIG. 1 in a closed position.

FIG. 5A is a top cross sectional view of the CO₂ delivery system of FIG. 1 in an open position.

FIG. 5B is a top cross sectional view of the CO₂ delivery system of FIG. 1 in a closed position.

FIG. 6A is a perspective view of another example CO₂ delivery system in an open position.

FIG. 6B is a perspective view of the CO₂ delivery system of FIG. 6A in a closed position.

FIG. 7A is a cross sectional view of the cap device of the CO₂ delivery system of FIG. 6A in an open position.

FIG. 7B is a cross sectional view of the cap device of the CO₂ delivery system of FIG. 6A in a closed position.

FIG. 8A is a top view of the CO₂ delivery system of FIG. 6A in an open position.

FIG. 8B is a top view of the CO₂ delivery system of FIG. 6A in a closed position.

FIG. 9A is a perspective view of an example adaptor for a CO₂ delivery system.

FIG. 9B is a cross sectional view of the adaptor of FIG. 9A coupled to the cap device of the CO₂ delivery system of FIG. 1.

FIG. 9C is a cross sectional view of the adaptor of FIG. 9A coupling the cap device of the CO₂ delivery system of FIG. 1 and an example fluid receptacle.

FIG. 10A is a perspective view of another example adaptor for a CO₂ delivery system.

FIG. 10B is a cross sectional view of the adaptor of FIG. 10A.

FIG. 10C is a cross sectional view of the adaptor of FIG. 10A coupling the cap device of the CO₂ delivery system of FIG. 1 and another example fluid receptacle.

FIG. 11 is a cross sectional view of an example receptacle of another CO₂ delivery system with a tablet loader.

FIG. 12A is a cross sectional view of another example CO₂ delivery system with a drinking straw in a CO₂ delivery position.

FIG. 12B is a cross sectional view of the CO₂ delivery system of FIG. 12A in a drinking position.

FIG. 12C is a cross sectional view of the CO₂ delivery system of FIG. 12A in a closed position.

FIG. 13 is a cross sectional view of another CO₂ delivery system.

FIG. 14 illustrates another example CO₂ delivery system in accordance with some embodiments.

FIG. 15 illustrates another example CO₂ delivery system in accordance with some embodiments.

### DETAILED DESCRIPTION

This document provides methods and materials for compensating for or treating hypocapnia. For example, this document provides methods and materials for delivering CO₂ to a human to treat/compensate for hypocapnia and/or to reduce the symptoms of hypocapnia. Symptoms of hypocapnia that can be reduced (e.g., rapidly reduced) as described herein include, without limitation, dizziness, visual disturbances, anxiety, muscle cramps, tetany, cutaneous signs of paresthesia (e.g., hands, feet, and/or mouth), nausea, headache, difficulty concentrating, imbalance as well as other neurological signs and symptoms, chest tightness, chest pain, bronchoconstriction, and epigastric distress. In some cases, the methods and materials provided herein can be used to treat hyperventilation and/or respiratory alkalosis. In some cases, the methods and materials provided herein can have the ability to use CO₂ enriched breathing gas as a modality for enhanced treatment of individuals who suffer from carbon monoxide poisoning. In some cases, this can be achieved by enhancing ventilation, carbon monoxide gas washout, and/or tissue oxygen delivery. Using CO₂ enriched breathing gas can be more effective as compared to mere oxygen or air inhalation.

Any appropriate method can be used to identify a person having hypocapnia, hyperventilation, and/or respiratory alkalosis. For example, clinical signs and symptoms as described above and/or a decrease in CO₂ (e.g., measured by end tidal CO₂ measurement, arterial, venous, or mixed venous blood gases, and/or direct exhaled gas analysis with capnometry or capnography) can be used to identify a human having hypocapnia. Additional changes that can be seen with acute hypocapnia can include low serum and urine phosphorus, low serum potassium, and/or low serum magnesium. In some cases, a human can self-identify when hypocapnia, hyperventilation, and/or respiratory alkalosis is present based on over breathing and/or being in an environment with reduced air pressure such as a high altitude when mountain climbing or flying. Exposure of a human to high altitude may result in adaptive increases in ventilation, which results in various degrees of hypocapnia. The methods and materials described herein can be used to detect hypocapnia, to correct hypocapnia, and/or to improve performance by adding CO₂ to the breathing air of the human.

In some cases, a capnic challenge test provided herein can be used to identify a human having hypocapnia. As described herein, a mammal (e.g., a human) can be assessed, prior to testing, for baseline symptoms and baseline measurements. The baseline symptoms can include, without limitation, items such as chest pain, feeling tense, blurred vision, dizziness, confusion, irregular breathing, shortness of breath, chest tightness, tingling fingers, stiff fingers or arms, tight feeling around mouth, cold hands or feet, palpitations, and/or anxiety feelings. Baseline symptoms can be those recorded on a Nijmegen questionnaire with a score of 23 being positive for disorders associated with decreased carbon dioxide as described elsewhere. Baseline measurements can include, without limitation, end tidal CO₂ recordings, blood pressure (e.g., orthostatic blood pressure), blood oxygen saturation levels, pulse rate, endothelial function, brachial tonometry, spirometry, and galvanic skin resistance. Any appropriate method can be used to measure end tidal CO₂. For example, a capnometer (e.g., a Masimo Capnometer) can be used to measure end tidal CO₂. Any appropriate method can be used to measure blood oxygen saturation levels and pulse rates. For example, an oximeter (e.g., a Masimo Oximeter) can be used to measure blood oxygen saturation levels and pulse rates.

Once baseline symptoms and baseline measurements are obtained, the CO₂ level within the mammal can be reduced over a period of time. For example, the CO₂ level within the mammal can be reduced by from about 10 mmHg to about 20 mmHg over a period of time from about 1 minute to about 10 minutes (e.g., from about 1 minute to about 5 minutes, from about 60 seconds to about 180 seconds, or from about 90 to about 120 seconds). For example, the CO₂ level within the mammal can be reduced from about 40 mmHg (e.g., 35 mmHg-45 mmHg) to about 20 mmHg (e.g., 15 mmHg-25 mmHg) over a period of time from about 1 minute to about 10 minutes (e.g., from about 1 minute to about 5 minutes, from about 60 seconds to about 180 seconds, or from about 90 to about 120 seconds). Any appropriate method can be used to reduce the CO₂ level within a mammal. For example, maximum voluntary ventilation can be used to reduce CO₂ levels within a mammal. The inhaled gas can be room air, 100% oxygen, or other oxygen rich mixtures.

In some cases, an increased intensity of ventilation over about 10 to 15 minutes can be used to achieve about 10 mmHg to about 20 mmHg reduction in CO₂ levels to demonstrate alterations in cerebral function simulating, for example, high altitude. Such a technique can be used as an educational tool for pilots and mountaineers. In the educational setting, pilots and mountaineers can be trained to learn and recognize the symptoms of hypocapnia. High inhaled oxygen concentration can cause increase in ventilation, which in turn will result in hypocapnia and decreased performance. In one example during training, the inhaled breathing gas mixture can be room air or, in some cases, 100% oxygen. The inhaled gas triggers excess ventilation resulting in hyperoxic hypocapnia, which is especially insidious as the subject perceives to have adequate oxygenation based on the inhaled gas, when the tissue oxygen delivery is markedly impaired by hypocapnic vasoconstriction and a left shift of the oxyhemoglobin dissociation curve.

While in a state of reduced CO₂, the mammal (e.g., the human) can be assessed for one or more symptoms and measurements such as any of the symptoms or measurements assessed at baseline. For example, the mammal can be assessed for one or more symptoms such as chest pain, feeling tense, blurred vision, dizziness, confusion, irregular breathing, shortness of breath, chest tightness, tingling fingers, stiff fingers or arms, tight feeling around mouth, cold hands or feet, palpitations, and/or anxiety feelings. Examples of measurements that can be assessed while the mammal is in a state of reduced CO₂ can include, without limitation, end tidal CO₂ recordings, blood pressure (e.g., orthostatic blood pressure), blood oxygen saturation levels, pulse rate, endothelial function, brachial tonometry, spirometry and skin resistance.

After assessing the mammal for symptoms and/or measurements while the mammal is in a state of reduced CO₂, the level of CO₂ can be replenished within the mammal. Any appropriate method can be used to restore CO₂ levels to their normal levels. For example, the methods and materials provided herein can be used to increase CO₂ levels within a mammal. In some cases, oral supplementation of CO₂ via an effervescent formulation can be used to increase CO₂ levels within a mammal. As the level of reduced CO 2 is being increased, or from about 60 seconds to about 300 seconds after baseline CO 2 levels are restored, the mammal (e.g., the human) can be assessed for resolution of one or more symptoms identified during the state of reduced CO₂. For example, a mammal that experienced one or more symptoms such as chest pain, feeling tense, blurred vision, dizziness, confusion, irregular breathing, shortness of breath, chest tightness, tingling fingers, stiff fingers or arms, tight feeling around mouth, cold hands or feet, palpitations, and/or anxiety feelings, during the state of reduced CO₂ can be assessed to determine if those symptoms resolved upon restoring baseline CO₂ levels. In some cases, measurements such as end tidal CO₂ recordings, blood pressure (e.g., orthostatic blood pressure), blood oxygen saturation levels, pulse rate, endothelial function, brachial tonometry, spirometry and skin resistance can be assessed as baseline CO₂ levels are being restored or after baseline CO₂ levels are restored.

Any symptoms that appeared during a state of reduced CO₂ and resolved during a return to baseline CO₂ levels can be attributed to hypocapnia (e.g., hypocapnia resulting from hyperventilation) and/or acute respiratory alkalosis. In such cases, a mammal (e.g., a human) identified as having hypocapnia and/or respiratory alkalosis via a capnic challenge test provided herein can be treated as described herein. In some cases, a mammal (e.g., a human) identified as having hypocapnia and/or respiratory alkalosis via a capnic challenge test provided herein can be treated, alternatively or additionally, with other therapeutic measures used to optimize breathing.

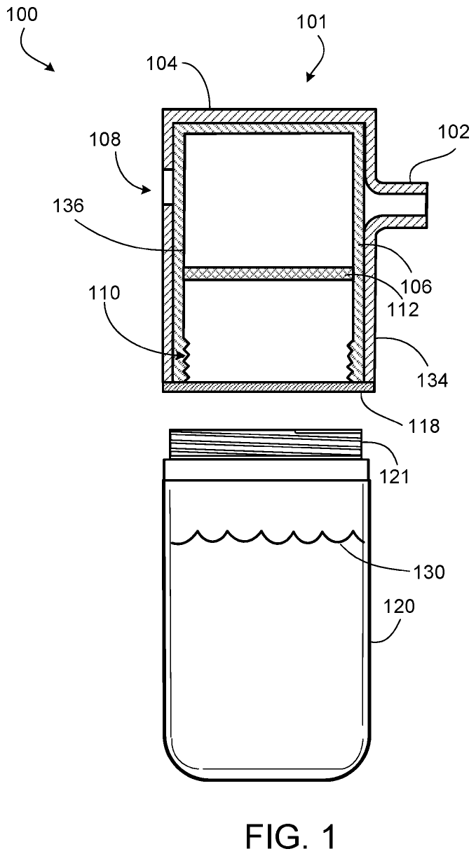

Once identified as having hypocapnia, hyperventilation, and/or respiratory alkalosis, a user can utilize a CO₂ delivery system to aid in delivering CO₂ to the lungs of the user. FIG. 1 depicts a CO₂ delivery system 100 that can be used to increase the amount of CO₂ inhaled by the user and delivered to the user's lungs. The CO₂ delivery system 100 includes a fluid receptacle 120 and a cap device 101 that is configured to be coupled to the fluid receptacle 120. The cap device 101 includes an outer shell 104 and an inner shell 106 that is nested within the outer shell 104.

The outer shell 104 of the cap device 101 includes a mouthpiece 102. The mouthpiece 102 is positioned on the sidewall 134 of the outer shell 104 and defines an opening through the sidewall 134 of the outer shell 104. The mouthpiece 102 is sized to be easily positioned within the mouth of a user. The mouthpiece 102 can be placed within the user's mouth and the user can form a seal around the outside of the mouthpiece 102 with her lips. Once the user has placed the mouthpiece 102 in her mouth, the user can breathe through the cap device 101 into and out of the fluid receptacle 120 by inhaling and exhaling through the mouthpiece 102.

The outer shell 104 includes a vent 108 that extends through the sidewall 134 of the outer shell 104. As will be described in further detail herein, the vent 108 allows gas to pass into and out of the cap device 101 as the users breathes through the mouthpiece 102, which ensures that a vacuum is not created within a fluid receptacle 120 that is coupled to the cap device 101 when a user breathes through the mouthpiece 102.

Still referring to FIG. 1, the cap device 101 includes threads 110 on an inner surface of the inner shell 106 that removably couple the cap device 101 to a fluid receptacle 120. For example, the fluid receptacle 120 can include threads 121 on an outside surface of the fluid receptacle 120 that are configured to releasably engaging with the threads 110 of the cap device 101.

The cap device 101 also includes a sealing element 118 positioned along an outer edge of the cap device 101 below the threads 110. The sealing element 118 is configured to form a seal with an outer surface of a fluid receptacle 120 coupled to the cap device 101 in order to prevent fluid from leaking out of the fluid receptacle 120 around the cap device 101. The sealing element 118 can be formed of rubber or any other suitable elastomeric material for sealing the cap device 101 to the fluid receptacle 120.

The cap device 101 includes a mesh 112 positioned within and spanning across the interior opening of of the cap device 101. The mesh 112 is configured to prevent any solids contained within a fluid receptacle coupled to the cap device 101 (e.g., fluid receptacle 120) from passing through the mouthpiece 102 of the cap device 101. The mesh 112 can be formed of any suitable metal or polymeric material.

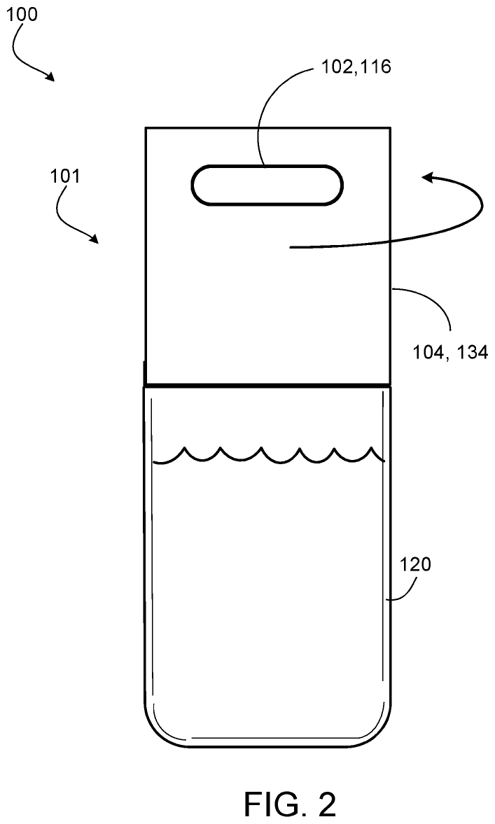

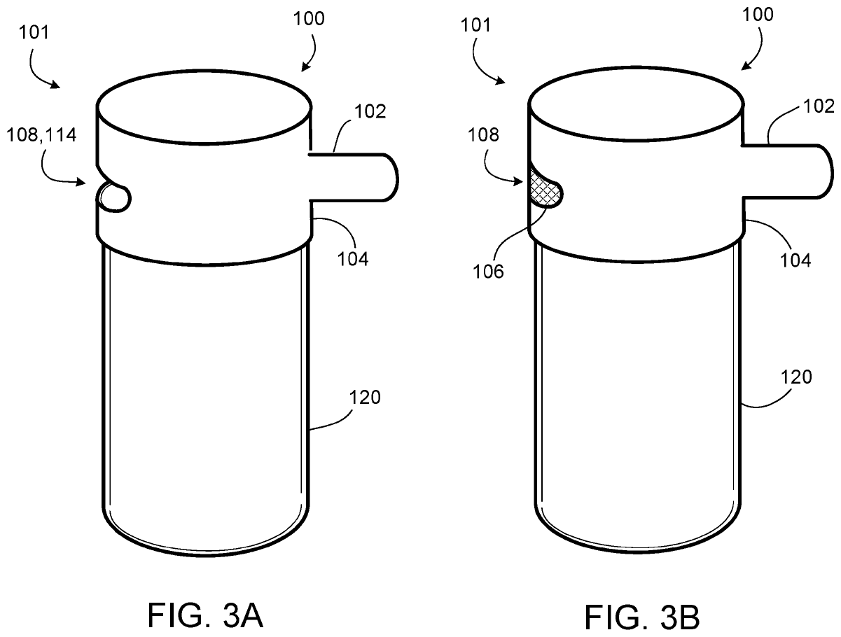

The outer shell 104 of the cap device 101 is rotatably coupled about the inner shell 106 such that the outer shell 104 can be rotated relative to the inner shell 106 between an open position, as depicted in FIGS. 2, 3A, 4A, and 5A, and a closed position, as depicted in FIGS. 1, 3B, 4B, and 5B. In some implementations, the outer shell 104 of the cap device includes ridges, bumps, or other means to aid the user in rotating the outer shell 104 relative to the inner shell 106 in order to open and close the cap device 101. When the cap device 101 is placed in the open position, fluid can flow through the mouthpiece 102 and vent 108, and when the cap device 101 is placed in the closed position, fluid is prevented from flowing through the mouthpiece 102 and vent 108.

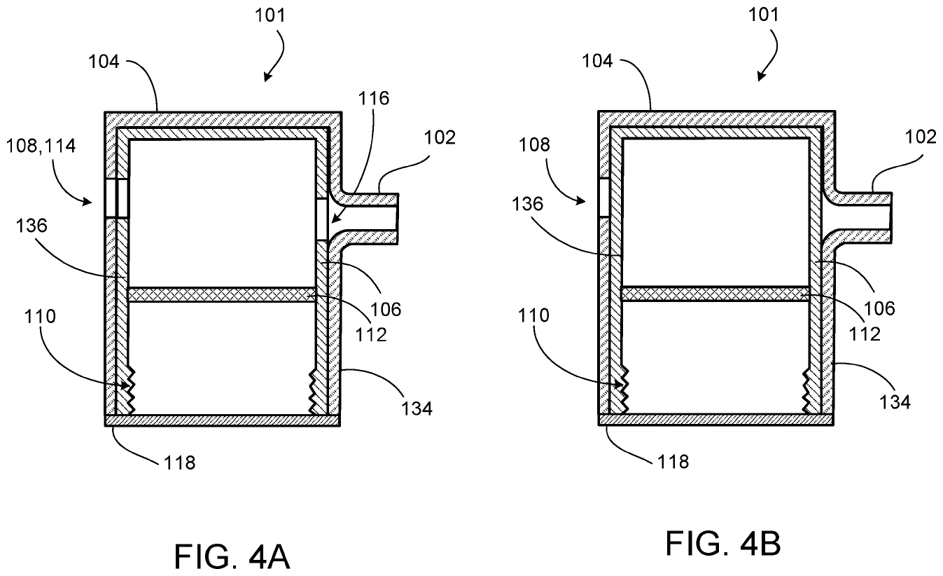

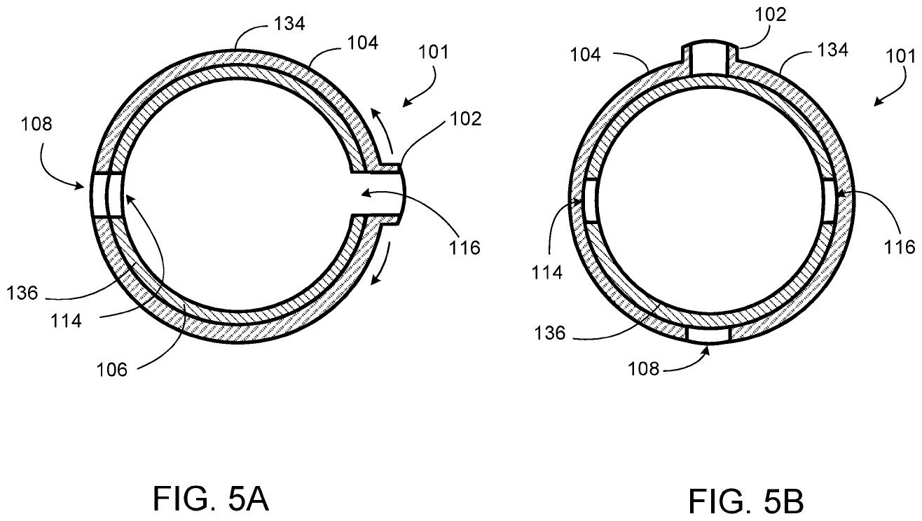

FIGS. 2, 3A, 4A, and 5A depict the cap device 101 in an open position. As can be seen in FIGS. 4A and 5A, the inner shell 106 of the cap devices includes a first opening 114 and a second opening 116 through a sidewall 136 of the inner shell 106. The outer shell 104 of the cap device 101 can be rotated relative to the inner shell 106 to align the vent 108 with the first opening 114 and align the mouthpiece 102 with the second opening 116, as depicted in FIGS. 4A and 5A. By aligning the vent 108 and mouthpiece 102 with the first and second openings 114, 116, the vent 108 and mouthpiece 102 are fluidly coupled with the interior of the cap device 101 and the fluid receptacle 120 coupled to the cap device 101. As a result, gas, such as CO₂, can flow into and out of the fluid receptacle 120 through the vent 108 and mouthpiece 102. By rotating the outer shell 104 of the cap device 101 into an open position in which the mouthpiece 102 is aligned with an opening 116 in the inner shell 106 of the cap device, a user is able to breathe into and out of the fluid receptacle 120 using the mouthpiece 102. In addition, by rotating the outer shell 104 of the cap device to align the vent 108 with an opening 114 through the inner shell 106, gas can pass into and out of the cap device 101 through the vent 108 as the users breathes through the mouthpiece of the cap device 101, which ensures that a vacuum is not created within the fluid receptacle 120 coupled to the cap device 101 when a user breathes through the mouthpiece 102 of the cap device 101. As will be explained in further detail herein, by breathing into and out of a fluid receptacle 120 using the cap device 101, the user inhales CO₂ and the inhaled CO₂ is provided to the user's lungs.

FIGS. 1, 3B, 4B, and 5B depict the cap device 101 in the closed position, in which fluid is prevented from flowing through the mouthpiece 102 and the vent 108. For example, as can be seen in FIGS. 4B and 5B, in order to move the cap device 101 from an open position (as depicted in FIGS. 4A and 5A) into a closed position (as depicted in FIGS. 4B and 5B), the outer shell 104 of the cap device 101 is rotated relative to the inner shell 106 of the cap device 101 such that the vent 108 and mouthpiece 102 are offset from (i.e., not aligned with) the openings 114, 116 through the inner shell 106. As a result, the sidewall 136 of the inner shell 106 covers the openings of the vent 108 and mouthpiece 102 and prevents fluid (e.g., liquid or gas) contained within the fluid receptacle 120 from flowing out of the fluid receptacle 120 through the vent 108 or mouthpiece 102.

A method of using the CO₂ delivery system 100 depicted in FIG. 1 to deliver CO₂ to a user will now be described.

Upon determining that a user is experiencing hypocapnia, hyperventilation, and/or respiratory alkalosis, the user prepares the CO₂ delivery system 100 for use by filling the fluid receptacle 120 partially with a liquid 130 (e.g., water), placing a composition formulated to release CO₂ inside the fluid receptacle 120 and in contact with the liquid 130 contained within the fluid receptacle 120, positioning the cap device 101 into the closed position (e.g., as depicted in FIGS. 1,3B, 4B, and 5B), and coupling the cap device 101 to fluid receptacle 120 by threading the threads 110 of the cap device 101 onto the threads 121 of the fluid receptacle 120. Threading the cap device 101 onto the fluid receptacle 120 brings the sealing element 118 of the cap device 101 into contact with an outer surface of the fluid receptacle 120, which prevents fluids (e.g., CO₂ gas or liquid 130) from flowing out of the fluid receptacle 120 around the cap device 101.

Once the solid composition formulated to release CO₂ for inhalation is wetted by the liquid 130 contained within the fluid receptacle 120, CO₂ is released from the composition, and the CO₂ travels upwards towards the top of the fluid receptacle 120 towards the cap device 101. Example compositions for releasing CO₂ that can be used in conjunction with the CO₂ delivery system 100 are described in U.S. Patent Application Publication No. 2020/0121875, which is incorporated by reference in its entirety herein.

Once the composition for releasing CO₂ has been wetted by the liquid 130 in the fluid receptacle 120 and the cap device 101 has been coupled to the fluid receptacle 120, the user can rotate the outer shell 104 of the cap device 101 relative to the inner shell 106 of the cap device 101 to align the vent 108 with a first opening 114 through the inner shell 106 and align the mouthpiece 102 with a second opening 116 through the inner shell 106, which places the cap device 101 in an open position (e.g., as depicted in FIGS. 2, 3A, 4A, and 5A) by fluidly coupling the vent 108 and mouthpiece 102 with the interior of the cap device 101 and the fluid receptacle 120.

Once the cap device 101 is in the open position with the vent 108 and mouthpiece 102 aligned with the openings 114, 116 through the inner shell 106, the user can place the mouthpiece 102 in her mouth, form a seal around the outside of the mouthpiece 102 with her lips, and begin breathing through her mouth in order to inhale CO₂ contained within the fluid receptacle 120 through the mouthpiece 102. As the user breathes through the mouthpiece 102, she also exhales CO₂ into the fluid receptacle 120 through the mouthpiece 102. The CO₂ exhaled by the user through the mouthpiece 102 into the fluid receptacle 120 can be re-inhaled by the user through the mouthpiece 102 and provided to the user's lungs as the user continues breathing through the mouthpiece 102. As the user breathes through the mouthpiece 102, the mesh 112 within the cap device 101 traps any solid particles (e.g., solid particles of the composition formulated to release CO₂) and prevents the solid particles from being inhaled by the user through the mouthpiece 102.

Whenever the user completes or temporarily stops her treatment, the user can remove the mouthpiece 102 from her mouth and close the cap device by rotating the outer shell 104 of the cap device 101 relative to the inner shell 106 of the cap device 101 until the vent 108 and mouthpiece 102 are no longer aligned with the openings 114, 116 through the inner shell 106 (e.g., as depicted in FIGS. 1, 3B, 4B, and 5B). In the closed position, the sidewall 136 of the inner shell 106 blocks the openings of the vent 108 and mouthpiece 102 to prevent any gas, such as CO₂, or liquid, such as liquid 130, from flowing out of the fluid receptacle 120 through the mouthpiece 102 or vent 108. As previously discussed, the sealing element 118 of the cap device 101 prevents any gas or fluid from leaking from the fluid receptacle 120 around the cap device 101. By placing the cap device 101 in the closed position when the user is not performing treatment, the CO₂ gas and liquid 130 contained within the fluid receptacle 120 is prevented for unintentionally flowing out of the fluid receptacle 120 and can be used later for additional treatment.

At the end of treatment, the user can also unthread the cap device 101 from the fluid receptacle 120 and discard the liquid 130 and any remaining solids from the fluid receptacle 120. Once the liquid 130 and any remaining solids have been discarded from the fluid receptacle 120, the cap device 101 can be reattached to the fluid receptacle 120 via threads 110, 121 and be placed in a closed position, as described above.

While certain embodiments have been described, other embodiments are possible.

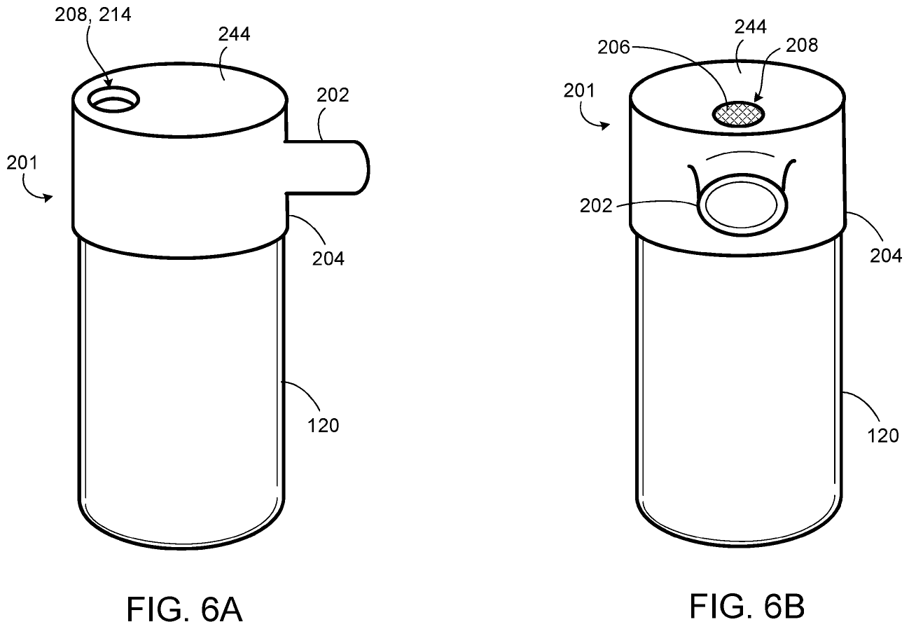

For example, while the vent 108 of the cap device 101 has been described as extending through a sidewall 134 of the outer shell 104, in some implementations the vent 208 of the cap device 201 extends through a top surface 244 of the outer shell 204, as depicted in FIGS. 6A-8B. Similar to cap device 101, the cap device 201 depicted in FIGS. 6A-8B includes an outer shell 204 and an inner shell 206 that is nested within the outer shell 204. A mouthpiece 202 is positioned on a sidewall 234 of the outer shell 204 of the cap device 201 and the cap device 201 includes a corresponding opening 216 through the sidewall 236 of the inner shell 206 of the cap device 201. Cap device 201 also includes a mesh 212 positioned within and spanning across the interior opening of the cap device 201, threads 210 to releasably couple a fluid receptacle (e.g., fluid receptacle 120 of FIG. 1) to the cap device 201, and a seal 218 positioned along a bottom edge of the cap device 201.

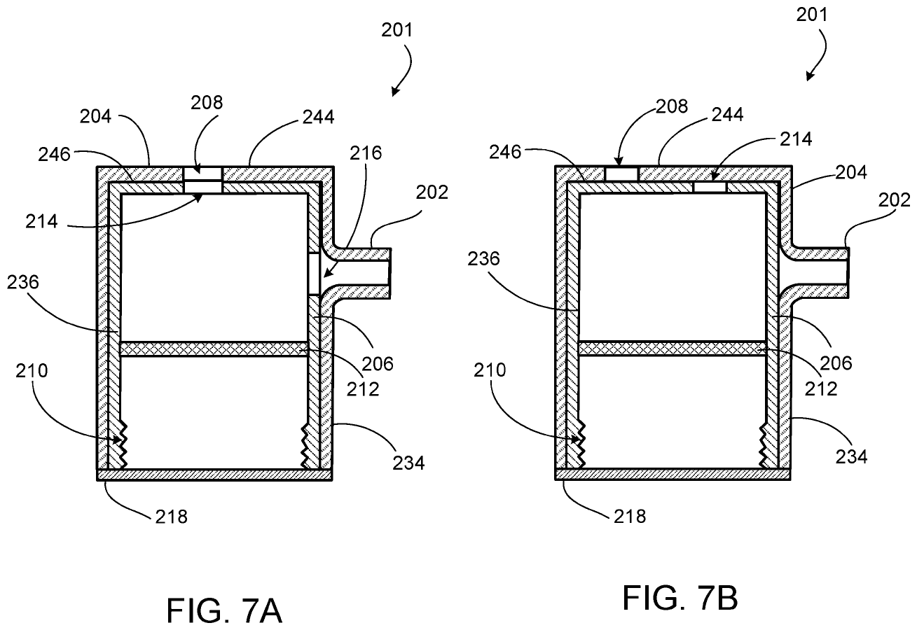

As can be seen in FIGS. 7A and 7B, the vent 208 of the cap device 201 extends through a top surface 244 of the outer shell 204 and the cap device includes a corresponding opening 214 that extends through a top surface 246 of the inner shell 206 of the cap device 201. Similar to vent 108 of cap device 101, the vent 208 allows gas to pass into and out of the cap device 201 as the users breathes through the mouthpiece 202 of the cap device 201, which ensures that a vacuum is not created within a fluid receptacle that is coupled to the cap device 201 when a user breathes through the mouthpiece 202 of the cap device 201.

Similar to cap device 101, the outer shell 204 of the cap device 201 is rotatably coupled about the inner shell 206 of the cap device 201 such that the outer shell 204 can be rotated relative to the inner shell 206 between an open position, in which fluid can flow through the mouthpiece 202 and vent 208, and a closed position, in which fluid is prevented from flowing through the mouthpiece 202 and vent 208.

In some implementations, the outer shell 204 of the cap device includes ridges, bumps, or other means to aid the user in rotating the outer shell 204 relative to the inner shell 206 in order to open and close the cap device 201.

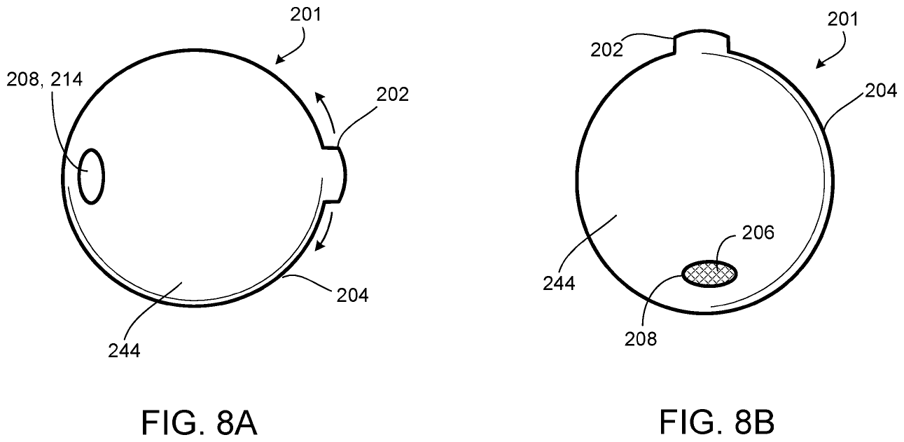

FIGS. 6A, 7A, and 8A depict the cap device 201 in an open position. As can be seen in FIGS. 7A and 8A, the outer shell 204 can be rotated relative to the inner shell 206 to align the vent 208 with the first opening 214 through the top surface 246 of the inner shell 206 and align the mouthpiece 202 with the second opening 216 through the sidewall 236 of the inner shell 206. By aligning the vent 208 and mouthpiece 202 with the first and second openings 214, 216 through the inner shell 206, the vent 208 and mouthpiece 202 are fluidly coupled with the interior of the cap device 201 and any fluid receptacle coupled to the cap device 101. As a result, gas, such as CO₂, can flow into and out of the fluid receptacle coupled to the cap device 201 through the vent 208 and mouthpiece 202 as a user breathes into and out of the fluid receptacle through the mouthpiece 202.

FIGS. 6B, 7B, and 8B depict the cap device 201 in the closed position, in which fluid is prevented from flowing through the mouthpiece 202 and vent 208. For example, as can be seen in FIG. 7B, in order to move the cap device 201 from an open position (as depicted in FIGS. 6A, 7A, and 8A) into a closed position (as depicted in FIGS. 6B, 7B, and 8B), the outer shell 204 of the cap device 201 is rotated relative to the inner shell 206 of the cap device 201 such that the vent 208 is offset from (i.e., not aligned with) the opening 214 through the top surface 246 of the inner shell 206 and the mouthpiece 202 is not aligned with the opening 216 through the sidewall 236 of the inner shell 206. As a result, the top surface 246 of the inner shell 206 covers the opening of the vent 208 and the sidewall 236 of the inner shell 206 covers the opening of the mouthpiece 202, which prevents fluid (e.g., liquid or gas) contained within a fluid receptacle coupled to the cap device 201 from flowing out of the fluid receptacle through the vent 208 or mouthpiece 202.

In addition, while the cap device 101 of the CO₂ delivery system 100 has been described as being directly coupled to a fluid receptacle (e.g., using corresponding sets of threads 110, 121), in some implementations, the cap device 101 can be coupled to fluid receptacles using an adaptor that couples to both the cap device 101 and the fluid receptacle.

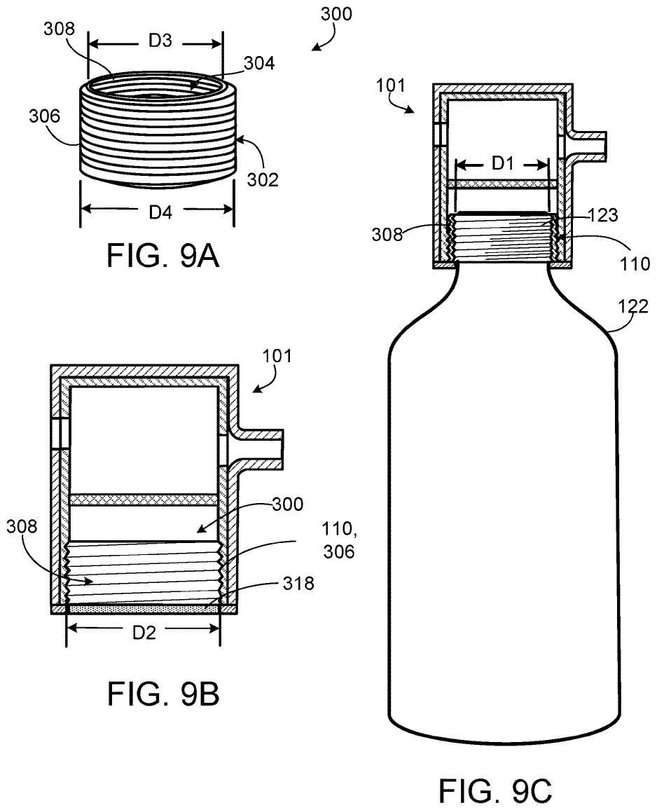

For example, referring to FIGS. 9A-9C, the cap device 101 can be coupled to an alternate fluid receptacle 122 using an adaptor 300. As can be seen in FIGS. 9B and 9C, the diameter D1 of the threaded portion 123 of the fluid receptacle 122 is smaller than the diameter D2 of the threaded portion 110 of the cap device 101. As a result, the fluid receptacle 122 cannot be directly coupled to the cap device 101 using the threads 110 of the cap device 101. Rather, as depicted in FIG. 9C, the fluid receptacle 122 is coupled to the cap device 101 using an adaptor 300 that is configured to releasably engage the threads 110 of the cap device 101 and the threads 123 of the fluid receptacle 122. As can be seen in FIG. 9A, the adaptor 300 includes a first set of threads 306 positioned on the outer surface 302 of the adaptor 300 and a second set of threads 308 positioned on an inner surface 304 of the adaptor. The first set of threads 306 is configured to releasably engage the threads 110 of the cap device 101 and the second set of threads 308 is configured to releasably engage the threads 123 of the fluid receptacle 122. In addition, the inner diameter D3 of the adaptor 300 corresponds to the diameter D1 of the threaded portion 123 of the fluid receptacle 122 and the outer diameter D4 of the adaptor 300 corresponds to the diameter D2 of the threaded portion 110 of the cap device 101. The adaptor 300 also includes a sealing element 318 that contacts and forms a fluid seal with the outer surface of the fluid receptacle 122 when the adaptor 300 is coupled to the fluid receptacle 122 in order to prevent fluid from leaking out of the fluid receptacle 122 around the adaptor 300.

In order to couple the fluid receptacle 122 to the cap device 101, the adaptor 300 is threaded onto the fluid receptacle 122 by mating the threads 308 on the inner surface 304 of the adaptor 300 with the threads 123 of the fluid receptacle 122. Once the adaptor 300 is coupled to the fluid receptacle 122, the adaptor 300 and the fluid receptacle 122 can be coupled to the cap device 101 by threading the threads 306 on the outer surface 302 of the adaptor 300 onto the threads 110 of the cap device 101. In some implementations, the adaptor 300 is threaded onto the cap device 101 before threading the adaptor 300 onto the fluid receptacle 122.

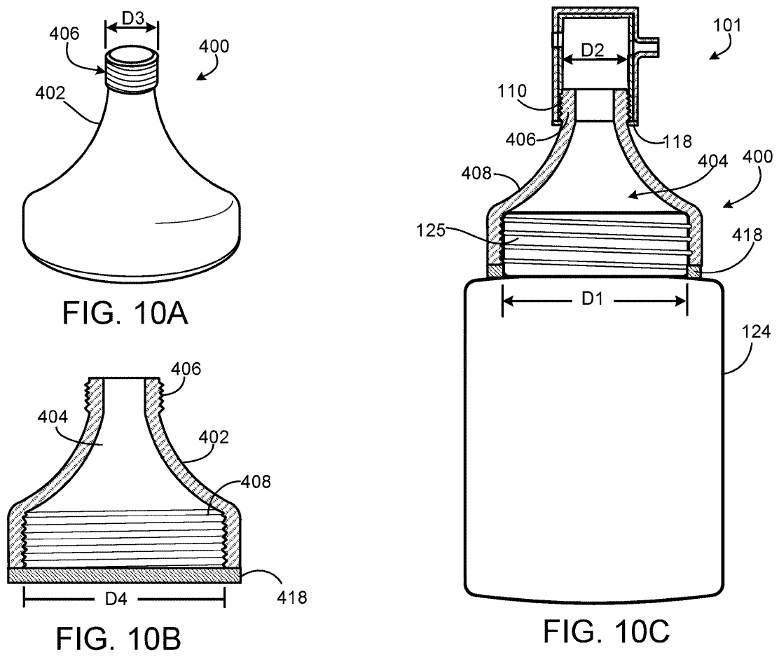

FIGS. 10A-10C depict another example adaptor for coupling an alternate fluid receptacle 124 to the cap device 101. As can be seen in FIG. 10C, the diameter D1 of the threaded portion 125 of the alternate fluid receptacle 124 is larger than the diameter D2 of the threaded portion 110 of the cap device 101. As a result, the fluid receptacle 124 cannot be directly coupled to the cap device 101 using the threads 110 of the cap device 101. Rather, as depicted in FIG. 10C, the fluid receptacle 124 is coupled to the cap device 101 using an adaptor 400 that is configured to releasably engage the threads 110 of the cap device 101 and the threads 125 of the fluid receptacle 124. As can be seen in FIGS. 10A and 10B, the adaptor 400 includes a first set of threads 406 positioned on the outer surface 402 of the adaptor 400 and a second set of threads 408 positioned on an inner surface 404 of the adaptor 400. The first set of threads 406 is configured to releasably engage the threads 110 of the cap device 101 and the second set of threads 408 is configured to releasably engage the threads 125 of the fluid receptacle 124. In addition, the outer diameter D3 of the portion of the adaptor 400 that includes the first set of threads 406 corresponds to the diameter D2 of the threaded portion 110 of the cap device 101 and the inner diameter D4 of the portion of the adaptor 400 that includes the second set of threads 408 corresponds to the diameter D1 of the threaded portion 125 of the fluid receptacle 124. As can be seen in FIGS. 10B and 10C, the adaptor 400 also includes a sealing element 418 that contacts and forms a fluid seal with the outer surface of the fluid receptacle 124 when the adaptor 400 is coupled to the fluid receptacle 124 in order to prevent fluid from leaking out of the fluid receptacle 124 around the adaptor 400.

In order to couple the fluid receptacle 124 to the cap device 101, the adaptor 400 is threaded onto the fluid receptacle 124 by mating the threads 408 on the inner surface 404 of the adaptor 400 with the threads 125 of the fluid receptacle 124. Once the adaptor 400 is coupled to the fluid receptacle 124, the adaptor 400 and the fluid receptacle 124 can be coupled to the cap device 101 by threading the threads 406 on the outer surface 402 of the adaptor 400 onto the threads 110 of the cap device 101. In some implementations, the adaptor 400 is threaded onto the cap device 101 before threading the adaptor 400 onto the fluid receptacle 124.

The fluid receptacle 120, 122, 124 coupled to the cap device 101 can be any type of suitable receptacle for containing a fluid and can be any suitable size, fluid-containing volume, and/or configuration. In some cases, the fluid-containing volume of the fluid receptacle is between 8 fluid ounces and 40 fluid ounces. In some cases, the fluid receptacle 120, 122, 124 is a plastic water bottle. For example, the fluid receptacle 120, 122, 124 can be a water bottle with a fluid-containing volume of 8 ounces, 10 ounces, 12, ounces, 16.9 ounces, 20 ounces, and so on.

In some implementations, the cap device 101 is formed of silicone and is collapsible to make the system 100 more compact. For example, the cap device 101 can include a bottom portion containing threads 110 and a top portion containing the vent 108 and mouthpiece that are each formed of a stiffer material, and the other portions the inner and outer walls 104, 106 extending between the top and bottom portions of the cap 101 can be formed of a silicone material that can be collapsed downwards towards the threads 110 when the system 100 is not in use. As a result, the cap device 101 and overall system 100 can be placed in a more compact position when not in use.

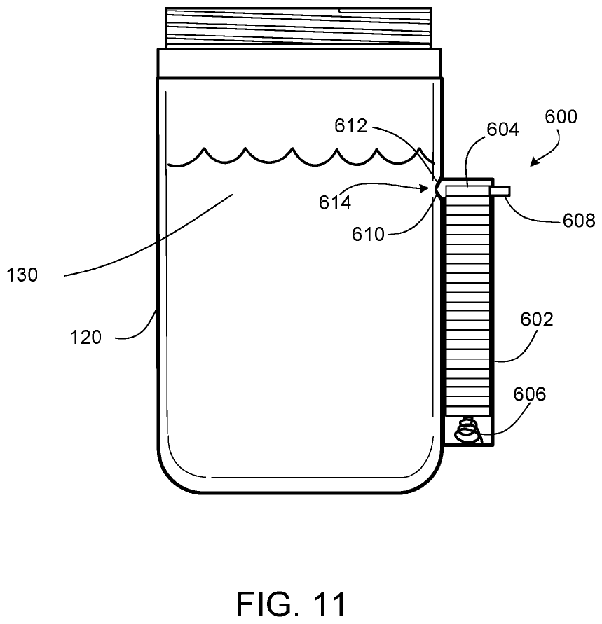

Referring to FIG. 11, in some implementations, the CO₂ delivery system 100 includes a tablet loading system 600 for placing a composition formulated to release CO₂ inside the fluid receptacle 120 and in contact with the liquid 130 contained within the fluid receptacle 120. The tablet loading system 600 includes a tablet housing 602 the receptacle 120 and fluidly isolated from the liquid 130 contained within the receptacle. The tablet housing 602 and is configured to receive one or more tablets 604 of the solid composition formulated to release CO₂. A spring 606 is positioned at the base of the tablet housing 602 and is configured to advance the tablets 604 inside the tablet housing 602 upwards toward the top of the tablet housing 602. A button 608 is positioned on the side of the tablet housing 602 that can be operated by a user to place a solid composition tablet 604 contained within the tablet housing 602 into contact with the liquid 130 in the receptacle 120. For example, a user can press the button 608 inwards toward the receptacle 120, which causes the button 608 to apply a force onto the solid composition tablet 604 adjacent the button 608 and forces the tablet 604 out of the tablet housing 602 through a pair of rubber flaps 610, 612 covering an opening 614 in the receptacle. When closed, the rubber flaps 610, 612 form a seal to prevent fluid from entering the tablet housing 602. Once the solid composition tablet 604 has been forced out of the tablet housing 602 through the rubber flaps 610, 612, the solid composition tablet 604 comes into contact with and is wetted by the liquid 130 contained within the fluid receptacle 120, which causes CO₂ to be released from the composition. A formulation of an effervescent tablet 604 can include the addition of vitamins and/or electrolytes to fortify the liquid 130 in which the tablet 640 is dissolved. In some embodiments, a coating can be included on the effervescent tablets 604 (similar to a coating on extended release medications) which would prevent tablets 604 in the tablet loader 604 from absorbing moisture.

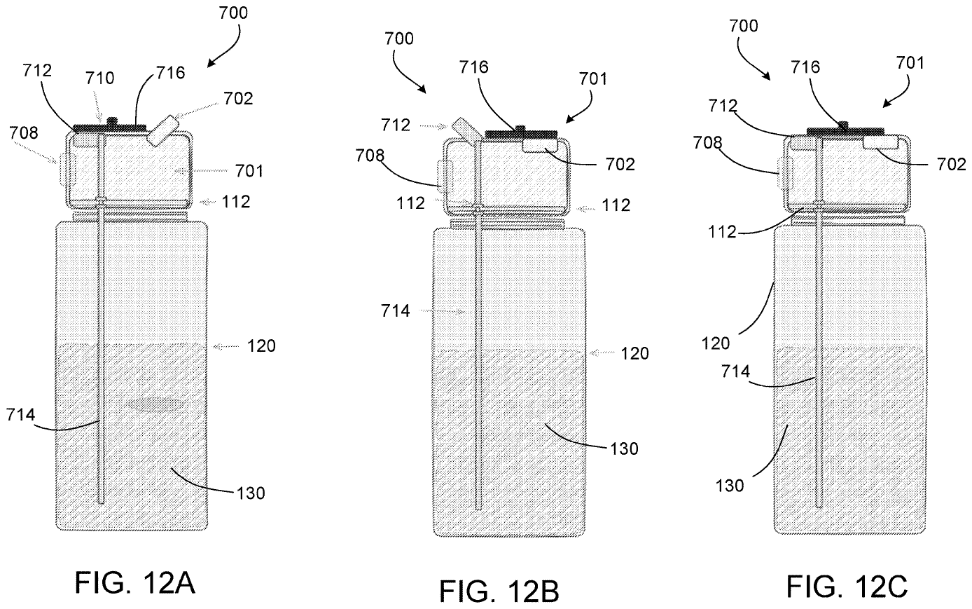

In some implementations, the CO₂ delivery system includes a drinking straw that enables the user to drink the liquid 130 contained within the receptacle 120. For example, as can be seen in FIGS. 12A-12C, a CO₂ delivery system 700 includes cap device 701 can include a drinking spout 712 that is fluidly coupled to a drinking straw 714 that extends through the mesh 112 in the cap device 701 into the liquid 130 contained within the receptacle 120.

When the user is using the CO₂ delivery system 700 to inhale CO₂, a slider 716 of the cap device 701 is positioned to cover and fluidly seal the drinking spout 712 and expose a mouthpiece 702 that can be used to inhale CO₂ contained inside the receptacle 120, as depicted in FIG. 12A. The process of generating and inhaling CO₂contained inside the receptacle 120 using the mouthpiece 702 is substantially similar to the process described with respect to FIGS. 1-10C. When the user is using the CO₂ delivery system 700 to drink the liquid contained within the receptacle, the slider 716 of the cap device 701 is positioned to cover and fluidly seal the mouthpiece 702 and expose the drinking spout 712, as depicted in FIG. 12B. Once the drinking spout 712 is exposed, the user can place her lips around the drinking spout 712 and draw the liquid 130 up through the straw 714 using drinking spout 712. When the user is not using the CO₂ delivery system 700 to inhale CO₂ or drink the liquid 130 inside the receptacle 120, the slider 716 is positioned to cover and fluidly seal both the mouthpiece and the drinking spout 712, as depicted in FIG. 12C. As a result, when the slider is in the “closed” position depicted in FIG. 12C, the liquid 130 is prevented from escaping the receptacle through either the drinking spout 712 or the mouthpiece 702.

When the user is using the CO₂ delivery system 700 to inhale CO₂ or drink the liquid 130 inside the receptacle, a vent 708 can be opened (for example pushed inwards) to allow ambient air into the cap device 701 to relieve pressure within the receptacle 120 and prevent the creation of a vacuum within the CO₂ delivery system 700 to inhale CO₂. Conversely, when the user is not using the CO₂ delivery system 700 to inhale CO₂ or drink the liquid 130 inside the receptacle, the vent 708 can be closed to prevent liquid 130 from escaping through the vent 708.

In addition, while the CO₂ delivery system 100 has been described as being used by placing a solid composition formulated to release CO₂ inside the fluid receptacle 120 and wetting the composition with a liquid 130 contained inside the fluid receptacle 120, in some implementations, the CO₂ delivery system 100 can be used without a solid composition formulated to release CO₂ or a liquid 130. For example, upon determining that the user is experiencing hypocapnia, hyperventilation, and/or respiratory alkalosis, a user would prepare the CO₂ delivery system 100 for use by simply coupling the cap device 101 to a fluid receptacle 120 that does not contain any liquid or CO₂-releasing composition, and rotating the outer shell 104 of the cap device 101 relative to the inner shell 106 of the cap device 101 to align the vent 108 with a first opening 114 through the inner shell 106 and align the mouthpiece 102 with a second opening 116 through the inner shell 106. Once the cap device 101 is in the open position with the vent 108 and mouthpiece 102 aligned with the openings 114, 116 through the inner shell 106, the user can place the mouthpiece 102 in her mouth, form a seal around the outside of the mouthpiece 102 with her lips, and begin breathing through the mouthpiece 102 into and out of the fluid receptacle 120. As the user breathes through the mouthpiece 102, she exhales CO₂ into the fluid receptacle 120 through the mouthpiece 102. The CO₂ exhaled by the user through the mouthpiece 102 into the fluid receptacle 120 can be re-inhaled through the mouthpiece 102 and provided to the user's lungs as the user continues breathing through the mouthpiece 102. The increased amount of dead space ventilation generated by the user breathing into and out of the fluid receptacle 120 compared to when the user breathes into and out of open atmosphere results in an increased amount of CO₂ being provided to the user's lungs. In some cases, the dead space volume provided by the fluid receptacle 120 ranges from 16 ounces to 18 ounces.

In addition, while the CO₂ delivery system 100 has been described as being used by placing a solid composition formulated to release CO₂ inside the fluid receptacle 120 and wetting the composition with a liquid 130 contained inside the fluid receptacle 120, in some implementations, the CO₂ delivery system 100 can be used with a solid composition that does not require any wetting in order to release CO₂ (i.e., without a the use of a liquid 130 within the fluid receptacle 120). For example, one or more dry adsorbent chemicals pre-saturated with CO₂, such as zeolite can be placed in the receptacle 120 and can be heated to a specified temperature using a heating element inside the receptacle 120 to cause the dry chemicals to release (desorb) CO₂ for inhalation by the user via the mouthpiece 102, 202.

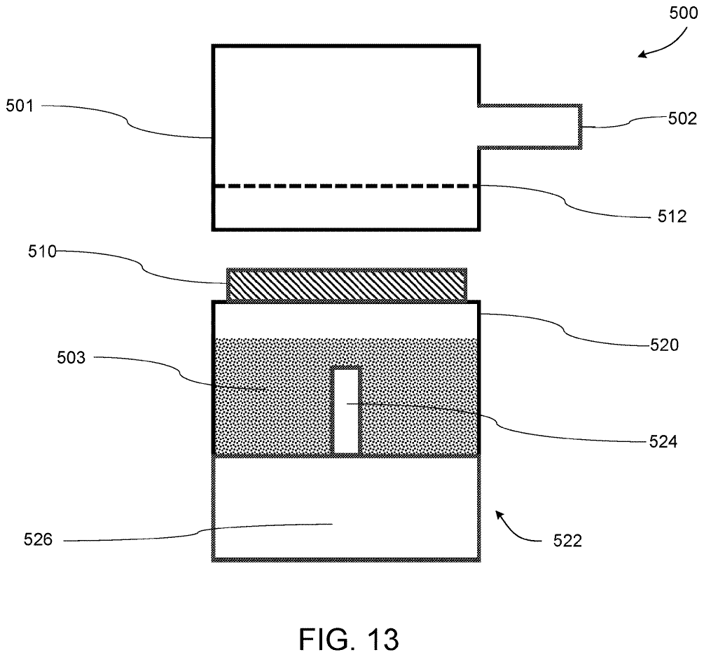

An example CO₂ delivery system 500 for use with a dry chemical composition for CO₂ generation is depicted in FIG. 13. The CO₂ delivery system 500 includes a dry chemical receptacle 520 for dry adsorbent composition 503 and a cap device 501 configured to releasably couple to the receptacle, for example, through a threaded connection. The cap device 501 includes a mouthpiece 502 that is sized to be easily positioned within the mouth of a user. The user can breathe through the cap device 501 into and out of the dry chemical receptacle 520 by inhaling and exhaling through the mouthpiece 102 in order to inhale CO₂ generated by heating the dry adsorbent composition 503 inside the receptacle 520. The cap device 501 also includes high efficiency filter screen 512 that prevents any of the dry chemical absorbent 503 from being inhaled by the user.

The receptacle 520 can include a quick-release latch 510, such as threads, a twist-lock latch, clamps, or similar, to allow the receptacle 520 to be removed quickly from the cap device 501. The receptacle 520 defines a void space to contain the dry adsorbent composition 503. The dry adsorbent composition 503 contained within the receptacle 520 can be in loose, pelletized, or containerized form. For example, in some implementations, the dry adsorbent composition 503 is contained within pre-formed pellets that are placed inside the receptacle 520, as depicted in FIG. 13. In some implementations, the dry adsorbent composition 503 is contained within a disposable cartridge that is placed inside the receptacle 520. The dry adsorbent composition 503 can include one or more zeolites and/or any other dry chemicals that have an ability to absorb CO₂.

The receptacle 520 includes heating system 522 to heat the dry adsorbent composition 503 to cause the dry adsorbent composition 503 to release its stored CO₂. The heating system 522 includes a heating element 524. The heating element 524 may take any suitable physical form, such as a central post, as depicted in FIG. 13.

The receptacle 520 also contains a reservoir 526 for batteries or fuel to power the heating system 522 and heat the heating element 524. Any suitable type of heating system 522 can be used to heat the dry adsorbent composition 503. For example, in some implementations, the heating system 522 used to heat the dry adsorbent composition 503 is an electric heating system that includes one or more batteries and a resistive element. In some implementations, the reservoir 526 includes a charging port to recharge one or more batteries contained within the reservoir 526 and used to heat the heating element 524. In some implementations, the reservoir 526 can be opened or otherwise accessed to replace one or more batteries contained within the reservoir 526. In some implementations, the heating system 522 includes a flame or catalytic reaction fueled by a combustible fuel, with the combustion kept separated from the stream of air inhaled by the user through the mouthpiece 502. In some implementations, the reservoir 526 of the heating system 522 is insulated in order to avoid excess heat transfer to the exterior surfaces of the receptacle 520 handled by the user.

A method of using the CO₂ delivery system 500 depicted in FIG. 13 to deliver CO₂ to a user will now be described.

Upon determining that a user is experiencing hypocapnia, hyperventilation, and/or respiratory alkalosis, the user prepares the CO₂ delivery system 500 for use by placing a dry adsorbent composition 503 formulated to release CO₂ inside the fluid receptacle 520 and coupling the cap device 501 to receptacle 520 via the quick release latch 510. Once the cap device 501 is coupled to the receptacle 520, the heating system 522 can be initiated to heat the heating element 524 heat the dry adsorbent composition 503 to a threshold temperature. In some implementations, the threshold temperature is a temperature above the ambient temperature in which the system 500 is being used.

Once the dry adsorbent composition 503 is heated to the threshold temperature, the dry adsorbent composition 503 releases (desorbs) its stored CO₂ into the receptacle 520 and the CO₂ travels upwards towards the top of the receptacle 520 towards the cap device 501. As the produced CO₂, travels towards the top of the receptacle 520, the CO₂ is diluted with the ambient air in the receptacle 520 to CO₂ levels that are safe for inhalation. In some implementations, the cap device 501 and/or the receptacle 520 includes a vent to dilute the CO₂ produced by heating the dry adsorbent composition 503 to a safe concentration for inhalation. In some implementations, the CO₂ produced by the dry adsorbent composition 503 is diluted via the air in the receptacle 520 to a CO₂ concentration of less than or equal to 5% CO₂ at sea level. In some implementations, the CO₂ delivery system 500 includes a barometric pressure sensor that can be used to determine an amount of released CO₂ that can be safely offered to the user for inhalation via the delivery system 500. For example, a larger amount of CO₂ admixture may be provided to the user at higher elevations compared to at lower elevations.

Once the dry adsorbent composition 503 has released its stored CO₂ and the produced CO₂ has been diluted with ambient air, the user can place the mouthpiece 502 in her mouth, form a seal around the outside of the mouthpiece 502 with her lips, and begin breathing through her mouth in order to inhale CO₂ contained within the receptacle 520 through the mouthpiece 502. As the user breathes through the mouthpiece 502, she also exhales CO₂ into the receptacle 520 through the mouthpiece 102. The CO₂ exhaled by the user through the mouthpiece 502 into the receptacle 520 can be re-inhaled by the user through the mouthpiece 502 and provided to the user's lungs as the user continues breathing through the mouthpiece 502. As the user breathes through the mouthpiece 502, the filter screen 512 within the cap device 501 traps any solid particles (e.g., solid particles of the dry adsorbent composition 503) and prevents the solid particles from being inhaled by the user through the mouthpiece 502.

At the end of treatment, the user can unthread the cap device 501 from the receptacle 520 and discard the depleted dry adsorbent composition 503 from the receptacle 520. Once the depleted dry adsorbent composition 503 has been discarded from the receptacle 520, the receptacle 520 can be refilled with fresh dry adsorbent composition 503 and the cap device 501 can be reattached to the receptacle 520 via quick latch mechanism 510.

While the CO₂ delivery system 500 depicts the mouthpiece 502 as being formed on a cap device 501 that is releasably coupled to the receptacle 520, in some implementations, the CO₂ delivery system 500 is a unibody design with a mouthpiece formed directed on the receptacle 520, the dry adsorbent composition 503 being provided into the receptacle 520 in a cartridge or pellets, and the CO₂ produced by heating the dry adsorbent composition 503 using the heating system 522 being diluted by ambient air entering the receptacle 520 through a vent (hole) through the receptacle 520.

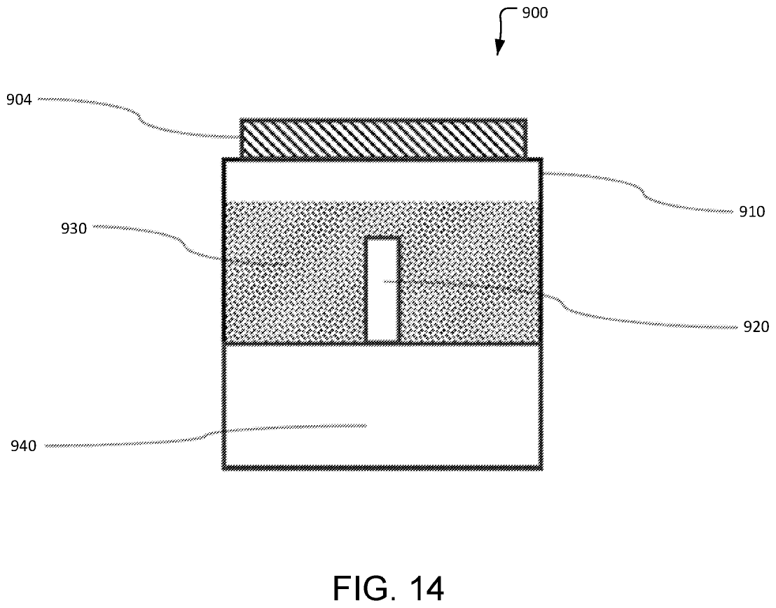

FIG. 14 depicts another example CO₂ delivery system 900 (or simply “system 900”) that utilizes zeolite as a CO₂ source. The system 900 includes a container 910 that can be smaller than a standard bottle (e.g., about one-half the height of a standard Nalgene® bottle in some embodiments). The container 910 can include a threaded opening 904 that can be coupled to any cover and breathing tube described above. A dry pellet zeolite in a filled cartridge 930 can be dropped into the bottle 910 at the time of use. The system 900 includes one or more heating element posts 920 throughout the cartridge 930. In some embodiments, a circumferential heating element is provided around the cartridge 930, and/or a bottom heating element can be included. In some embodiments, an electronics and battery package 940 is incorporated into the container 910. The electronics and battery package 940 can include an on/off switch with LED light and/or a USB charging port in some embodiments.

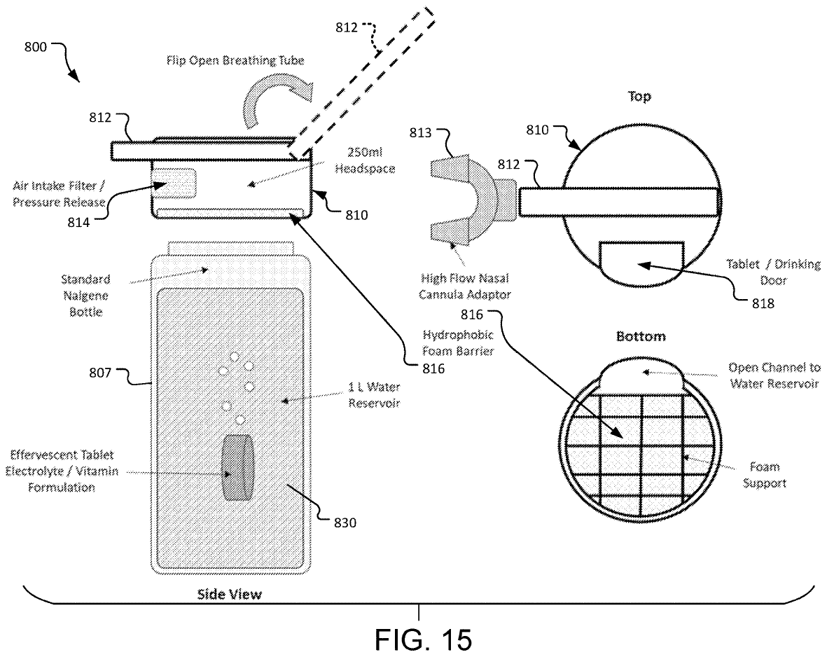

FIG. 15 depicts another example CO₂ delivery system 800 (or simply “system 800”). The system 800 includes a cap 810 that is configured to be releasably coupled to a standard bottle 807. The cap 810 includes a breathing tube 812 that is movably coupled to the main body of the cap 810. In the depicted embodiment, the breathing tube 812 is pivotably coupled to the main body of the cap 810. A breathing interface member, such as a nasal cannula adapter 813, can be removably attached to the breathing tube 812. In the depicted embodiment, the cap 810 also includes an air intake and pressure release 814 that can include a filter member. The cap 810 can also include a hydrophobic foam barrier 816 positioned between an upper air space in the cap and the bottle opening. In addition, the cap 810 can include an openable and closable door 818 that leads to an open passageway to the bottle (the open passageway is uninhibited/unblocked by the hydrophobic foam barrier 816). The door 818 can be placed in an open position in order to add a solid composition formulated to release CO₂ for inhalation into the bottle 807 and/or for a user to drink fluid 830 contained inside the bottle 807.

In addition, while the process of generating CO₂ by heating one or more dry adsorbent chemicals pre-saturated with CO₂ has been described as being performed using the CO₂ delivery systems 100, 500 depicted in FIGS. 1-15, other CO₂ delivery systems can be used to heat dry adsorbent chemicals pre-saturated with CO₂ and deliver CO₂ to a user. For example, more compact devices, such as pen-shaped devices similar to those used for vaping, could be used to heat dry adsorbent chemicals pre-saturated with CO₂ to generate CO₂ and deliver the generated CO₂ to a user for inhalation.

In some cases, a device designed to generate CO₂ gas as described herein (e.g., a CO₂ delivery system described herein) can be incorporated into an existing oxygen supply system such as an oxygen supply system of an aircraft. For example, a CO₂ delivery system as described herein can be coupled to and placed in line with an oxygen source (e.g., molecular sieve oxygen system, emergency oxygen system, bail out bottle, or oxygen generator) to provide CO₂ in addition to the emergency oxygen provided. For example, an oxygen system can include a compressed oxygen gas system, a chemical oxygen generation system, and/or a molecular sieve oxygen system.

The compositions and systems provided herein can be used to treat hypocapnia, to compensate for low levels of CO₂, to treat carbon monoxide intoxication, to enhance oxygenation, and/or to enhance performance and safety at high altitudes (e.g., at altitudes greater than 1500 m). The inhaled CO₂ provided as described herein can right shift oxygen-Hb dissociation curves, increase cerebral perfusion, enhance tissue oxygen delivery, increase cerebral tissue oxygen reserve time, enhance cognitive performance, enable dislodging of carbon monoxide (CO) from hemoglobin molecules, and/or mitigate deleterious hypocapnia. In some cases, the compositions and systems provided herein can be used to supply CO₂ for treatment of CO poisoning with oxygen, to enhance altitude hypoxia resistance (oxygen sparing), to provide emergency depressurization of aircraft, to provide a differential diagnosis of hypoxia vs. hypocapnia at altitude, and/or to provide field use to increase tissue oxygenation.

A formulation of the solid composition formulated to release CO₂ for inhalation can include the addition of vitamins and/or electrolytes to fortify the liquid (e.g., liquid 130) in which the solid composition is dissolved.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A system for delivering carbon dioxide (CO₂) to a user, the system comprising:
a fluid receptacle defining an interior space to contain a fluid; and
a cap device configured to be coupled to the fluid receptacle, the cap device comprising:
an inner shell;
an outer shell that is rotatably coupled about the inner shell;
a mouthpiece positioned on the outer shell;
a vent extending through the outer shell;
a first opening extending through the inner shell; and
a second opening extending through the inner shell.

2. The system of claim 1, wherein the cap device is configured to be positioned in an open position by rotating the outer shell relative to the inner shell to align the vent with the first opening and to align the mouthpiece with the second opening, wherein the mouthpiece is in fluid communication with the interior space of the fluid receptacle when the cap device is coupled to the fluid receptacle and in the open position.

3. The system of claim 1, wherein the cap device is configured to be positioned in a closed position by rotating the outer shell relative to the inner shell to offset the vent and the mouthpiece from the first opening and the second opening, wherein the mouthpiece is not in fluid communication with the interior space of the fluid receptacle when the cap device is coupled to the fluid receptacle and in the closed position.

4. The system of claim 1, wherein the mouthpiece is positioned on a sidewall of the outer shell and at least one of the first opening and the second opening extends through a sidewall of the inner shell.

5. The system of claim 1, wherein the vent is positioned on a sidewall of the outer shell and at least one of the first opening and the second opening extends through a sidewall of the inner shell.

6. The system of claim 1, wherein the vent is positioned on a top surface of the outer shell and at least one of the first opening and the second opening extends through a top surface of the inner shell.

7-23. (canceled)

24. A cap device configured to be coupled to a fluid receptacle for delivering carbon dioxide (CO₂) to a user, the cap device comprising:
an inner shell;
an outer shell that is rotatably coupled about the inner shell;
a mouthpiece positioned on the outer shell;
a vent extending through the outer shell;
a first opening extending through the inner shell; and
a second opening extending through the inner shell.

25. The cap device of claim 24, wherein the cap device is configured to be positioned in an open position by rotating the outer shell relative to the inner shell to align the vent with the first opening and to align the mouthpiece with the second opening.

26. The cap device of claim 24, wherein the cap device is configured to be positioned in a closed position by rotating the outer shell relative to the inner shell to offset the vent and the mouthpiece from the first opening and the second opening.

27. The cap device of claim 24, wherein the mouthpiece is positioned on a sidewall of the outer shell and at least one of the first opening and the second opening extends through a sidewall of the inner shell.

28. The cap device of claim 24, wherein the vent is positioned on a sidewall of the outer shell and at least one of the first opening and the second opening extends through a sidewall of the inner shell.

29. The cap device of claim 24, wherein the vent is positioned on a top surface of the outer shell and at least one of the first opening and the second opening extends through a top surface of the inner shell.

30-34. (canceled)

35. A method of delivering carbon dioxide (CO₂) to a user, the method comprising:
attaching a cap device to a fluid receptacle defining an interior space to contain a fluid, the cap device comprising a mouthpiece;
placing the cap device in an open position, wherein the mouthpiece is in fluid communication with the interior space of the fluid receptacle when the cap device is coupled to the fluid receptacle and in the open position; and
breathing into and out of the fluid receptacle through the mouthpiece of the cap device.

36. The method of claim 35, wherein:
the cap device comprises an inner shell and an outer shell rotatably coupled about the inner shell; and
placing the cap device in the open position comprises rotating the outer shell relative to the inner shell.

37. The method of claim 36, wherein:
the cap device comprises an opening extending through the inner shell; and
placing the cap device in the open position comprises rotating the outer shell relative to the inner shell to align the mouthpiece with the opening.

38. The method of claim 37, wherein:
the cap device comprises:
a vent extending through the outer shell; and
a second opening extending through the inner shell; and
placing the cap device in the open position comprises rotating the outer shell relative to the inner shell to align the vent with the second opening.

39-45. (canceled)

46. A method of delivering carbon dioxide (CO₂) to a user, the method comprising:
placing a dry carbon dioxide saturated composition that releases CO₂ when heated within a receptacle defining an interior space;
heating the dry carbon dioxide saturated composition inside the receptacle using a heating system at least partially disposed within the interior space to a threshold temperature to generate CO₂; and
breathing into and out of the receptacle through a mouthpiece fluidly coupled to the interior space.

47. The method of claim 46, further comprising attaching a cap device comprising the mouthpiece to the receptacle.

48. The method of claim 46, further comprising mixing the generated CO₂ with ambient air inside the receptacle to produce a diluted CO₂ gas for inhalation.

49. The method of claim 46, wherein the dry carbon dioxide saturated composition comprises one or more types of carbon dioxide saturated zeolites.
